(19) United States
(12) Patent Application Publication
(10) Pub. No.: US 2005/0232902 A1
(43) Pub. Date: Oct. 20, 2005

(54) Injectable bioartificial tissue matrix

(76) Inventor: Theodoros Kofidis, Hannover (DE)

Correspondence Address: BELL & ASSOCIATES, 416 FUNSTON ST., SUITE 100, SAN FRANCISCO, CA, 94118, US

(21) Appl. No.: 11/108,381

(22) Filed: Apr. 18, 2005

Related U.S. Application Data
(60) Provisional application No. 60/563,095, filed on Apr. 17, 2004.

(51) Int. Cl. A61K 45/00; C12N 5/06; C12N 5/08

(52) U.S. Cl. 424/93.7; 435/366; 435/364

(57) ABSTRACT

The present invention encompasses a liquid bioartificial tissue for restoring tissue and organ function to an injured or damaged organ in a human subject. The liquid bioartificial tissue is injected into a target organ and can significantly restore organ function within two weeks. The invention also encompasses a cell culture medium comprising ascorbic acid (or other free-radical scavengers and/or anti-oxidants) that is used for pre-treating transplantable cells prior to organ transplantation. Pre-treatment with ascorbic acid increases transplanted cell viability and colonization by nearly fifty-fold compared with untreated cells. The invention is particularly useful for treating ischemic heart damage following myocardial infarction.

The present application claims priority to U.S. Provisional Patent Application Ser. No. 60/563,095 entitled “Injectable Biosynthetic Tissue Matrix”, filed Apr. 17, 2004, which is herein incorporated by reference in its entirety for all purposes.

### FIELD OF THE INVENTION

The present invention relates to compositions and methods used to create an injectable bioartificial tissue matrix; more specifically to compositions and methods used to treat and restore heart myocardium following an infarction event. The invention also relates to compositions and methods used to increase the viability of cells that are injected into an organ, more specifically to using ascorbic acid as an adjuvant.

### BACKGROUND

The recent progress in harvest, culture and differentiation of embryonic stem (ES) cells has spurred research activity for tissue and organ restoration by cell transfer (Mummery, C., et al. (2002) J. Anat. 200: 233-242; Kehat, I., et al. (2001) J. Clin. Invest. 108: 407-414). Major concerns on the use of human embryonic stem cells limit their clinical potential, that is, differentiation into oncogenic phenotypes and the host response following implantation (Thomson, J. A., et al. (1998) Science 282: 1147; Xu, C., et al. (2001) Nature Biotechnol. 19: 971-974; Amit, M., et al. (2000) Dev Biol. 227: 271-278). Even though scientific protocols hold promise for their future use, their potential to survive, differentiate in vivo, and thereby improve organ function has not been sufficiently studied.

The heart is a potential target organ for ES cell transfer due to the terminal differentiation of the majority of cardiomyocytes and their limited capacity to regenerate (Pasumarthi, K. B. and Field, L. J. (2002) Circ. Res. 90: 1044-1054). Murray et al. have reported survival of a mixed population of human embryonic stem cells (hESC) in the healthy rat heart (Murray et al. (2003) American Heart Association summer summit, Salt Lake City, Aug. 12-16, 2003). This population of cells contained approximately 15% cells expressing cardiac markers in vitro and were not labelled prior to injection into the myocardium. Unique morphological characteristics such as their hypodense nucleus and their vacuolized cytoplasmic pattern distinguished them from the surrounding host myocardium. There was evidence of in vivo selection towards the cardiac phenotype, while the majority of the cells which did not express cardiac markers disappeared after 4 weeks. There are no successful reports of hESC transfer into myocardium. Furthermore, morphological evidence of in vivo dividing and differentiating hESC that assume the target organ-specific properties is still missing. Longitudinal studies of hESC-survival in a living recipient and evaluation of eventual cellular metastasis to distant parts of the recipient's body have yet to be published. The present invention provides a composition that results in superior in vivo ES cell survival in myocardium following myocardial ischemia and functional improvement of the host heart following intramyocardial injection.

Cell transfer for restorative purposes is primarily limited by early cell death. Many scientists have abandoned cell transfer procedures or abandoned using specific cell types (such as embryonic stem cells) because they identified massive cell death following cell transplantation. There is general agreement that at least 95% of the transplanted cells die following transplantation into the host target organ and therefore they cannot develop the desired effect. (See, for example, Etzion et al., (2001) Am. J. Cardiovasc. Drugs 1: 233-244.)

### CURRENT STATE OF THE ART

The sequence of events following severe myocardial ischemia due to obstruction of coronary flow can have detrimental effects on cardiac structure and function (Anversa, P., et al. (2002) J. Mol. Cell Cardiol. 34: 91-105). Myocardial cell necrosis is an irreversible process that can ultimately lead to heart failure. Innovative tissue engineering techniques developed to reconstitute organ function after a severe insult promise to diversify our approach to this condition but are associated with significant challenges. A major one is the distortion of cardiac geometry and structure, a crucial determinant of proper hemodynamic function. The scaffold's physical condition, its in vivo kinetics, and its suitability as an adequate microenvironment for the inoculated cells, are another limitations. Finally, sensitive primordial cells with questionable potential to survive in an area of a lesion, constitute a restriction of utmost significance.

The heart constitutes a complex helical structure (Buckberg, G. D., (2002) J. Thor. Cardiovasc. Surg. 124: 863-883) with significant local asymmetry and anisotropy. Variable portions of the left ventricle display distinct mechanical performance and microstructure. The contractions of the particular elements of all synchronized portions of the ventricle have to be orchestrated for maximal hemodynamic output. The vast array of biodegradable materials designed for implantation into the injured ventricular wall were not destined to achieve such a task (Cassell, O. C., et al. (2001) Ann. N.Y. Acad. Sci. 944: 429-442; Ozawa, T., et al. (2002) J. Thorac. Cardiovasc. Surg. 124: 1157-1164). Furthermore, most of the utilized biomaterials constitute single-component isotropic matrices, in which cells are seeded according to the rules of gravity, resulting in non-homogenous distribution and therefore inconsistent performance throughout the graft; the periphery of the graft which lies in culture medium or borders the host tissue is privileged in its blood or nutrient supply, as opposed to the core of the graft which is exposed to severe undersupply conditions (Robinson, K. A. and Matheny, R. G. (2002) Heart Surg. Forum 6: 8). The natural sequence of events is loss of viable donor cells and therefore loss of function. Furthermore, myocardial infarction frequently results in aneurysm formation, i.e. thinning of the affected left ventricular wall, and, according to the law of Laplace, an even more significant increase of circumferential wall stress. This results in a worse environment for the engrafted cells, with liberation of cytokines, derivates of the purine metabolism, free radical formation, and accordingly, more cell death.

As a result, the eventual improvement of cardiac function after replacement of portions of either ventricle is frequently ascribed to secondary angiogenesis activity triggered by the implanted grafts without clarity which mechanisms mediate such a response (inflammatory or rather a targeted paracrine effect) (Shimizu, T. (2002) Heart Surg. Forum 6: 4). A homogenously populated graft that would align itself to the intricate geometry of the remodeling tissue post-infarction and would not add to the load of the diseased area of has not yet been introduced.

The optimal type of cell to support and maintain the injured region of the heart is still controversial. Common sense implies that the ideal cell source would be one's own body to maximize the likelihood of survival and engraftment of the cells. There is a rich body of work with autologous bone marrow stem cells and autologous myoblasts with variable and questionable success. The lacking peripheral plasticity of the former and the limited intercalation of latter with their host counterparts restricts their potential for large-scale sustained myocardial restoration significantly.

Two recent decisive developments in cell science and microsurgery might help harness the potential of tissue engineering and propel efforts to restore myocardium. Of paramount importance, pluripotent embryonic stem cells isolated from the embryonic trophoblast have become easy to maintain and to purify in a more committed state. The remarkable potential of these cells to self-renew or give rise to a more differentiated progeny translates into high viability and promises survival in the hostile environment of an ischemic lesion. Green fluorescent protein (GFP)-based labelling of donor cells has been introduced to identify and track the in vivo fate of donor cells (Monosov, E. Z., et al. (1996) J. Histochem. Cytochem. 44: 581-589; Afting, M., et al. (2003) Tissue Eng. 9: 137-141). Simple and reliable methods of cell labelling prior to transplantation should be viewed as an essential part of studies which involve cell or tissue transfer.

Of note, several prior publications have disclosed using a liquid medium in which cultured cells are suspended ad the liquid is injected into a tissue in vivo. WO03024462A1 discloses a method of administering hematopoietic stem cells to a heart, whereby the stem cells differentiate into cardiac muscle cells thereby treating heart failure and improving cardiac function. U.S. Pat. No. 6,387,369 discloses is a method for producing cardiomyocytes in vivo by administering mesenchymal stem cells (MSCs) to the heart. These cells can be administered as a liquid injectable or as a preparation of cells in a matrix that is or becomes solid or semi-solid.

In addition to the above two publications, other publications disclose the use of ES cells that are injected into myocardium; use of adult hematopoietic stem cells injected into adult myocardium; and use of neonatal cardiomyocytes transplanted into adult myocardium. (See U.S. Pat. No. 6,534,052; Balsam, L. B., et al. (2004) Nature 428: 668-673; Min, et al. (2002) J. Appl. Physiol. 92: 288-296; Muller-Ehmsen J., et al. (2002) J. Mol. Cell Cardiol. 34: 107-116; Reffelmann, T., et al. (2003) Heart Fail. Rev. 8: 201-211; Reinlib, L. et al. (2000) Circulation. 101: 182; Judith A. Shizuru, J. A., et al. (2000) Proc. Natl. Acad. Sci. 97: 9555-9560.)

The extracellular matrix (ECM) of mammalian tissue comprises many proteins, including collagen, fibronectin, actin, vitronectin, members of the laminin, tenascin, and thrombospondin families, and a variety of proteoglycans. Recent studies have shown that fibronectin increases the mechanical performance of artificial tissue constructs comprising collagen (Gildner et al. (2004) Am. J. Physiol. Heart Circ. Physiol. Mar. 4, 2004 online publication, 10.1152/ajpheart.00859.2003).

Ascorbic acid (vitamin C) is involved in a number of biological processes. Originally identified as the agent necessary to prevent scurvy, it is now considered amongst the most important reducing agents (antioxidants) of the cell. It functions as a co-substrate in many oxido-reduction reactions, including, but not limited to, post-translational hydroxylation of proline in the formation of collagen, catecholamine synthesis by dopamine-β-monoxygenase, neutralize intracellular free radicals, such as trioxide or peroxide, and as an electron donor in the electron transfer chain in the absence of oxygen or in the presence of cyanide. It is also a chelating agent and facilitates the absorption of iron from the intestine.

A number of studies have shown that inclusion of ascorbic acid as an antioxidant and an adjuvant in a culture medium can result in enhanced growth and induction of differentiation in cell cultures. Of note, Buttery et al. show that differentiation of murine ES cells towards the osteoblast lineage can be enhanced by supplementing serum-containing media with ascorbic acid and other adjuvants (Buttery et al. (2001) Tissue Eng. 7: 89-99).

In addition, U.S. Patent Application Publication No. 2004/0030406 A1 discloses a method of incubating ascorbic acid with a “tissue equivalent” (an artificial gelled tissue culture composition) in preparation for transplantation. The applicants disclosed that incubating chondrocytes with ascorbic acid for three weeks in vitro resulted in an approximately ten-fold increase in cell viability; 2×10⁵ cells (starting number) incubated with 50 μg/ml ascorbic acid resulted in approximately 12×10⁵ viable cells in the gelled culture; a similar number of cells cultured in the absence of ascorbic acid resulted in 1.25×10⁵ viable cells. However, in separate experiments where the starting number of cells was 2×10⁶ cells, only 2.6×10⁶ cells remained viable after three weeks incubation with ascorbic acid. Applicants did not show whether the cells' viability remained following transplantation into a tissue in vivo. (See U.S. Patent Application Publication No. 2004/0030406, Pub. Date 12 Feb. 2004.)

A number of other reports note that ascorbic acid is used to promoted osteoblast differentiation from fat-derived or bone derived stem cells, or functional dopamine neurons from central nervous system-derived embryonic cells. (See Lee et al. (2003) Annals Plastic Surg. 50: 616-617; Gurevich et al. (2002) Tissue Eng. 8: 661-672; Kim et al. (2003) J. Neurochem. 85: 1443-1454; and U.S. Pat. No. 6,617,159 B1.)

Recent efforts, both experimentally and in human subjects, to treat advanced stages of disease, such as heart infarction or other types of organ failure, such as liver cirrhosis or renal failure, using stem cell transplantation, face major limitations. In many cases, the transplanted cells or tissue dies or becomes progressively less viable within days following transplantation into the host. Therefore the engraftment processes are impaired and the potential restorative role of the transplanted cells or tissues for the target organ's function and structure is limited.

There is a clear need for a culture medium in which ES cells can differentiate and proliferate following transplant into the infarcted myocardium in vivo. Various transplantable tissue matrices comprising ES cells or cardiomyocytes have been used in the past with poor outcomes, such as reduced cell viability, functionality, and regenerative ability.

### BRIEF DESCRIPTION OF THE INVENTION

The invention provides a composition that results in superior in vivo ES cell survival in myocardium following myocardial infarction and functional improvement of the host heart following intramyocardial injection.

The invention encompasses a liquid bioartificial tissue comprising stem cells in a liquid matrix. This liquid tissue can be introduced into an injured or damaged organ to provide a graft. The stem cells differentiate into cells appropriate for the target organ. The liquid matrix can comprise collagen but is not limited to that protein. The liquid can be introduced into a damaged organ by injection but is not limited to that method. The stem cells can be embryonic stem cells but are not limited to that lineage. The target organ can be the heart but is not limited to that tissue. The injury or damage can be infarction injury but is not limited to that damage.

The invention provides a bioartificial tissue comprising embryonic stem cell-derived cardiomyoblasts in a liquid collagen matrix that is introduced into infarcted myocardium. The embryonic stem cell-derived cardiomyoblasts can then differentiate and proliferate into the area surrounding the site of introduction thereby colonizing a region of the myocardium that is larger in area than the region originally introduced. The embryonic stem cells can be human embryonic stem cells but are not limited to that species. Other species include, but are not limited to, non-human primates embryonic stem cells, porcine embryonic stem cells, caprine embryonic stem cells, ovine embryonic stem cells, rodent embryonic stem cells, and mouse embryonic stem cells.

In use the liquid bioartificial tissue comprising human embryonic stem cell-derived cardiomyoblasts is introduced into an area of a target organ that has been injured or damaged. The liquid state of the bioartificial tissue allows the liquid and the cells to flow beyond the confines of the area of the injured or damaged tissue, thereby colonizing at least the full extent of the injured or damaged tissue. The liquid bioartificial tissue can also colonize a region of the organ beyond the full extent of the injured or damaged tissue.

The present invention also encompasses a method of preparing a cell culture matrix for injection into a target organ, the method comprising the steps of providing embryonic stem cells, culturing the stem cells to induce cardiomyoblast formation, mixing the embryonic stem cell-derived cardiomyoblasts with a medium comprising collagen, and injecting the mixture into an injured or damaged target organ. In an additional embodiment, the medium also comprises fibronectin.

Other compositions such as growth factors, angiogenic factors, anti-oxidants and nutrients may also be included in the cell culture matrix.

Mixing of the stem cells and other structural and biochemical components of the matrix may be done immediately prior to transplantation. In some embodiments, mixing of the components may be done no more than 30 minutes, 20 minutes, 10 minutes, 5 minutes, or 1 minute before transplantation. Mixing can be achieved by using a syringe having two compartments with a single delivery lumen that also serves as a mixing chamber. A variety of mixing syringes are known and are described, for example in U.S. Pat. Nos. 5,281,198; 5,549,381; 5,501,371; 5,122,117; 4,464,174; 4,159,570; 4,116,240; and 4,041,945. Such syringes may be adapted for use with the present invention.

The present invention further encompasses a method of restoring the function of an injured or damaged target organ, the method comprising the steps of providing embryonic stem cells, culturing the stem cells to induce cardiomyoblast formation, mixing the embryonic stem cell-derived cardiomyoblasts with a medium comprising collagen, injecting the mixture into the injured or damaged target organ, thereby restoring the function of the injured or damaged target organ.

The invention encompasses cell culture medium formulations comprising ascorbic acid to increase viability of transplanted cells in a target organ. The transplanted cells can be stem cells, but are not limited to that lineage. The target organ can be a heart but is not limited to that organ.

Ascorbic acid is not the only substance that can be used with this invention to increase the viability of the transplanted cells, and various other substances or combinations of substances may be used instead of or in addition to ascorbic acid, such as vitamin E, any anti-oxidants, any free-radical scavenging species, or α-lipoic acid (that aids the regeneration of vitamin C and vitamin E), or the like. When vitamin C or ascorbic acid is mentioned in this disclosure, it is to be understood that other molecules that perform the same of a similar function may equally be used.

In certain embodiments, ascorbic acid (or similar-functioning substances) may be included in the ES culture medium, and the ES cells exposed it ascorbic acid for up to 24 hours. The matrix comprising collagen, etc. is then mixed with the ES cells and the liquid bioartificial tissue injected to the transplantation site. In other embodiments, the ascorbic acid, or other components may be injected into the site of transplantation separately from the ES tissue matrix.

The invention further encompasses cell culture medium formulations comprising ascorbic acid to increase viability of transplanted cells in an injured or damaged target organ.

In an alternative embodiment, the invention encompasses a defined cell culture medium comprising ascorbic acid for culturing stem cells, whereby cultured cells are exposed to the medium prior to mixing the stem cells with the liquid matrix and are then transplanted to a target organ and that results in increased cell proliferation of the transplanted cells in the target organ.

In a further embodiment of the invention, the invention encompasses a defined cell culture medium comprising ascorbic acid whereby cultured cells that are exposed to the medium and then transplanted to a target organ results in increased cell viability of the transplanted cells in the target organ.

The invention also encompasses a method of using ascorbic acid (or other free-radical scavengers, anti-oxidants, α-lipoic acid or similar-functioning compounds) in a defined medium to increase viability of transplanted cells in a target organ. The transplanted cells can be stem cells, but are not limited to that lineage. The target organ can be a heart but is not limited to that organ. In certain applications, the target organ may be a heart damaged by myocardial infarction.

The use of a liquid bioartificial tissue offers significant advantages over the previously known compositions and methods. The liquid bioartificial tissue can be injected into an injured or damaged myocardium, such as following myocardial infarction, using minimally invasive procedures, such as using an endoscope device, without distorting the heart's architecture or structure. In addition, preparation of the liquid bioartificial tissue can be performed within a few minutes, and thus overcomes the time limitation of several weeks' culture to prepare a solid bioartificial tissue. The liquid state of the bioartificial tissue also has the advantage of being in a state to which other adjuvants are readily added, thereby allowing a physician or health worker to tailor the liquid bioartificial tissue to the patient's clinical needs. The liquid bioartificial tissue can be potentially effective for other organ restoration, such as liver, kidney, brain, bone, and reproductive organs.

### BRIEF DESCRIPTION OF THE DRAWINGS

The file of this patent contains at least one drawing executed in color. Copies of this patent with color drawing(s) will be provided by the U.S. Patent and Trademark Office upon request and payment of the necessary fee.

FIG. 1 shows a photograph of infarcted myocardium stained to show donor (red) and host (blue) collagen structures in tissue injected with bioartificial tissue comprising embryonic stem cells.

FIG. 2 shows a photograph of infarcted myocardium stained to show GFP-positive donor cells (green) that co-localize with DAPI (blue) and with connexin 43 (red).

FIG. 3 shows a graph that illustrates the percentage fractional shortening of the ventricular contractile cycle following different treatments.

FIG. 4 shows a photomicrograph of a cross-section of myocardial tissue at ×50 magnification. Control ES cells had been injected into the myocardium. The area of myocardial infarction damage and ischaemia is delineated by a white line. The area colonized by control, untreated ES cells is delineated by a yellow line. Bright green staining indicates the presence of exogenous cells.

FIG. 5 shows a photomicrograph of a cross-section of myocardial tissue at ×25 magnification. The ES cells had been pretreated for 24 hours prior to injection into the myocardium. The area of infarction damage and ischaemia is delineated by a white line. The area colonized by ascorbic acid-treated ES cells is delineated by a yellow line. Bright green staining indicates the presence of exogenous cells. Note that the intensity of the green staining differs at different magnifications.

FIG. 6 shows the green fluorescing donor cells that were injected within the liquid matrix and formed colonies in the host infarcted area. Red staining represents expression of connexin 43, a marker of cardiac differentiation (presence of gap junctions indicating potential communication between the cells), clearly indicating in vivo development of the cells towards the desired phenotype of the heart muscle cell. (Confocal colocalization; ×650; bar=100 μm)

FIG. 7 shows the increase of fractional shortening (FS, a measure of the heart's pump function and viability) following injection of the bioartificial mixture. This increase is superior to control experiments where only cells or only matrix were injected. (Key: Matrigel+Cells, Group I; Infarcted Control, Group II; Matrigel, Group III; Cells, Group IV; **:p<0.0001)

### DETAILED DESCRIPTION OF THE INVENTION

The embodiments disclosed in this document are illustrative and exemplary and are not meant to limit the invention. Other embodiments can be utilized and structural changes can be made without departing from the scope of the claims of the present invention.

As used herein and in the appended claims, the singular forms “a,” “an,” and “the” include plural reference unless the context clearly dictates otherwise. Thus, for example, a reference to “a cell” includes a plurality of such cells, and a reference to “an adjuvant” is a reference to one or more adjuvants and equivalents thereof, and so forth.

Bioartificial Tissue

The invention is drawn to bioartificial tissue that has utility and that is used to restore an injured or damaged organ in a mammal.

In one embodiment, the invention is a liquid bioartificial tissue that is used to repopulate and restore the myocardium of a heart injured or damaged by infarction.

In a preferred embodiment, the liquid bioartificial tissue comprises a solution of collagen molecules and embryonic stem cell-derived cardiomyoblasts. In an additional embodiment the liquid bioartificial tissue comprises fibronectin. The liquid state of the bioartificial tissue can let a health worker or physician introduce the bioartificial tissue into a target organ using an endoscope device or the like, thereby reducing the clinical complications associated with more invasive surgery techniques and methods. The health worker or physician can visually inspect and identify an area of a target organ using an endoscope comprising a miniature camera or the like. In a preferred embodiment, the area of the target organ comprises an area of disease, injury, or damage to that target organ. In a more preferred embodiment the area of disease, injury, or damage to that target organ is an area of the left ventricular myocardium that has undergone myocardial infarction.

The health worker or physician then introduces a needle suitable for injecting the liquid bioartificial tissue into the area of injury or damage using an endoscope device or the like. The health worker or physician connects the needle from the endoscope device to a mixing device, the mixing device comprising a first chamber holding the cultured embryonic stem cell-derived cardiomyoblasts and a second chamber holding a liquid collagen composition. In an additional embodiment of the invention, the second chamber holds a liquid collagen and fibronectin composition. The physician or health worker mixes the contents of the two chambers and introduces the resulting liquid bioartificial tissue comprising the mixed cells and liquid collagen compositions into the target tissue through the needle. In a preferred embodiment the liquid bioartificial tissue is injected into at least one area of the infarcted myocardium. In an alternative embodiment, the liquid bioartificial tissue is injected into at least two areas of the infarcted myocardium.

The liquid state of the bioartificial tissue allows the cells and collagen components to infiltrate an area of the infarcted myocardium to a greater extent that if the cells and collagen compositions were in a gel or solid state. When the liquid bioartificial tissue is introduced into a target tissue the collagen in the composition interacts with endogenous components of the myocardium and the collagen polymerizes to create a protein scaffold network with which cells and other proteins can interact. The protein scaffold network can also provide strength and stiffness to the infiltrated area of the myocardium thereby improving myocardial contractile forces that results in improved heart function.

In another alternative embodiment of the invention, the liquid bioartificial tissue is introduced into an alternative secondary target area of the individual before introducing the bioartificial tissue into the myocardium. In this alternative embodiment, the liquid bioartificial tissue also comprises proteins and other co-factors that can induce angiogenesis. Angiogenesis-inducing proteins are well known to those in the art, such, as but not limited to, vascular endothelial growth factor (VEGF), and the like. Co-factors can be signaling molecules such as the thyroid hormones T₃ and T₄ and the like. An exemplary alternative target area is the mesentery of the gut, which is rich in blood vessels. Another exemplary alternative target area is the renal fatty capsule. The liquid bioartificial tissue solidifies in one of the alternative target areas and is infiltrated by newly proliferating blood vessels. Following a predetermined period, the bioartificial tissue is resected from the surrounding alternative target tissue and is then introduced into a previously created cavity within the target tissue. The bioartificial tissue is therefore already vascularized and is amenable to the formation in vivo of an intercommunicating vasculature with the vascular system of the target organ. This increases the blood flow and consequent delivery of oxygen to the bioartificial tissue and that is no longer flowing from the vasculature originally dependent upon the blood supply from the coronary artery.

In another embodiment of the invention, the liquid synthetic tissue comprises stimulatory drugs, such as β-adrenergic agonists, microcircuits, and nanoparticles. In an alternative embodiment of the invention, the liquid bioartificial tissue also comprises neonatal or adult stem cells derived from the tissues of a human subject. In another embodiment, the neonatal or adult stem cells can be derived from the same human subject candidate for treatment with the liquid bioartificial tissue thereby reducing host-graft rejection.

Although the present disclosure generally discusses use of the liquid bioartificial tissue of the invention for restoring areas of an injured or damaged myocardium, the liquid bioartificial tissue may also be further used in a number of other clinically relevant applications. Such applications include, but are not limited to the following: abdominal surgery, gastrointestinal restorative surgery, pancreatic disease such as type I diabetes or cancer, renal failure, liver disease such as hepatitis, cirrhosis, or cancer, head injury, hemorrhagic stroke, neurological restorative surgery, muscle restorative surgery, reproductive disorders, and vascular surgery.

In addition, the liquid bioartificial tissue of may also be used in the treatment of severe sepsis, septic shock, the systemic inflammatory response associated with sepsis, rheumatological disease, eczema, psoriasis, contraction of tissues during wound healing, excessive scar formation during wound healing, organ transplant, or graft versus host disease.

In the case of organ transplant, the liquid bioartificial tissue can wherein the transplant is a corneal, kidney, heart, lung, heart-lung, skin, liver, gut or bone marrow transplant.

Ascorbic Acid as an Adjuvant in Restoration of Myocardium

The invention encompasses compositions and methods wherein ascorbic acid (or other free-radical scavengers and/or anti-oxidants, and/or α-lipoic acid) is added to a culture medium used to introduce ES-derived cardiomyocytes into a myocardium damaged by infarction.

Such anti-oxidants are, for example, but not limited to, dithiothreitol, 2-mercaptoethanol, or glutathione; antioxidant enzymes, such as, but are not limited to, superoxide dismutase (SOD), glutathion peroxidase (GPx), glutathion reductase (GSH), catalase, and the like. Free-radical scavenging species are, for example, but are not limited to, EPC-K1, a phosphate diester linkage of vitamins E and C, myricetin 3-O-alpha-rhamnopyranoside, (−)-epigallocatechin 3-O-gallate, (−)-epigallocatechin, (+)-gallocatechin, gallic acid, amifostine, and melatonin, pyruvate, catechins (including, but not limited to, epicatechin, epicatechin gallate, epigallocatechin, epigallocatechin gallate) and related compounds (methyl gallate, 4-methylcatechol, and 5-methoxyresorcinol), and the like.

In a preferred embodiment of the invention, ascorbic acid is included in a cell culture medium used to provide nutrients to a culture of cells that are treated for use for subsequent transplantation into a target organ. The ascorbic acid can be in the form of an acid, a hemicalcium salt, a phosphate sesquimagnesium salt, a sulphate dipotassium salt, a dehydro dimer, or any other chemical form. The cells are treated with the culture medium comprising ascorbic acid prior to transplantation into a target organ.

Different cell types have different transporter systems and some will take up ascorbic acid faster than others. Because of this, the duration of exposure of the cells to ascorbic acid may be variable. Sometimes a relatively short exposure is desirable, and the exposure time may be at least 20 minutes, at least 1 hour, or at least 2 hours. Generally exposure is up to about 24 hours using 1 mM ascorbic acid; but exposure may be for a period of between 4 hours and 96 hours, or between 8 hours and 72 hours, or between 16 hours and 48 hours, or between 24 hours and 36 hours. The concentration of ascorbic acid (or other free-radical scavengers and/or anti-oxidants) used in the medium may also be varied between about 0.01 mM to 10 mM, or from 0.05 mM to 5 mM, or from 0.1 mM to 1 mM. Different concentrations may be used depending on the other components of the culture medium. If one uses a complex culture medium containing bovine serum albumin (BSA) or similar complex biological components, then serum proteins may tend to adsorb active molecules, such as ascorbic acid, reducing the effective concentration. If using a simple, well-defined serum-free culture medium, then there will be fewer proteins to adsorb and sequester ascorbic acid molecules, making the effective concentration higher than when compared with a complex medium, despite that fact that the same amount of ascorbic acid is added to the medium. In one preferred embodiment, the cells are treated for about twenty-four hours prior to transplantation with a medium containing 1 mM ascorbic acid. The cells are then transplanted by transfer into the target organ. In another preferred embodiment the target organ is a heart, but the target organ is not limited to that organ. In still another preferred embodiment the cells are transferred by injection into the target organ, but transfer of the cells is not limited to that method.

In use, the inclusion of ascorbic acid (or other free-radical scavengers and/or antioxidants) in the culture medium results in increased growth and viability of the transferred cells in the target organ. The known properties of ascorbic acid can be attributed to this effect. As noted above, ascorbic acid functions as a co-substrate in many oxidation-reduction reactions, such as post-translational hydroxylation of proline residues in the formation of collagen; catecholamine synthesis by dopamine-beta-monoxygenase; neutralizing intracellular free radicals, such as trioxide or peroxide; and as an electron donor in the electron transfer chain in the absence of oxygen (or in the presence of cyanide). Ascorbic acid is also a factor in the biosynthesis of noradrenaline that is the precursor to adrenaline which is important in heart muscle cell function. Each one of these functions, either alone, or in combination in the transplanted myocardium, can increase the regeneration of intercellular collagen matrices or networks, can increase the intramuscular pool of catecholamines that regulate myocardial function, and/or can allow electron transfer through the cytochrome system in the absence of oxygen (such as in ischaemic tissues).

The ascorbic acid-treated cells can become large conglomerates of cells in the injected tissue and thereby can restore major portions of a damaged heart muscle when compared with tissue injected with untreated cells. The proportion of host tissue target organ restored by the grafted cultured cells can be evaluated by a measure termed the “graft/infarct ratio”. The graft/infarct ratio is the ratio of the portion of the ‘dead tissue’ area of the target organ that is restored and/or replaced by transplanted cells of bioartificial tissue. The ratio is determined by dividing a measured area of the restored and/or replaced area by the total measured area of ‘dead tissue’. In addition, a measure or growth and/or of viability can be determined by other methods known in the art. Such methods include, staining of tissue slices with trypan blue to determine extent of dye exclusion, determining nucleic acid uptake of tritiated thymidine into proliferating cells, and staining of tissue slices with toluidin blue to determine extent of dye uptake.

The injectable bioartificial matrix does not only miniaturize experimentation in restorative heart surgery, but makes restorative intervention on the beating heart. Novel surgical approaches for the therapy of heart disease should be less invasive and associated with less surgical trauma and in-hospital stay for the patient. All these benefits could be reached through the approach introduced in the work accomplished by Kofidis et al. (2005, Circulation, in press).

### EXAMPLES

The invention will be more readily understood by reference to the following examples, which are included merely for purposes of illustration of certain aspects and embodiments of the present invention and not as limitations.

### Example I

### Preparation and Injection of ES-Derived Cardiomyocytes into Small Animal Model of Myocardial Infarction

Undifferentiated Green Fluorescent Protein (GFP)-labeled mouse ES cells (2×10⁶) were seeded in BD MATRIGEL matrix (BD Biosciences, Bedford Mass.). The ES cell suspension in MATRIGEL was maintained at a constant 37° C. The resulting cell suspension was the liquid bioartificial tissue.

Lewis rats (150-200 g) were used in all experimental procedures. The Lewis rat is used as a heterotopic heart transplant model. In this example, the left anterior descending coronary artery (LAD) was ligated to create an intramural left ventricular pouch (infarcted area of the myocardium). The ES cells suspended in 0.125 ml MATRIGEL were injected in the resulting infarcted area within the pouch and the suspension became solid within a few minutes after transplantation. Five recipient groups were studied: transplanted healthy hearts (Group I), infarcted control animals (Group II), matrix recipients alone (Group III), the study group which received matrix plus cells (Group IV), and a group which received ES cells alone (Group V). Two weeks later, the animals were subjected to echocardiography to visualize the extent of colonization by the cardiomyocytes.

Rats were transferred in a portable anesthesia chamber and kept under inhalative Isoflurane anesthesia for the duration of intramural injection of the bioartificial tissue, which took place immediately before sacrifice. The chests were shaved and the animals placed in recumbent position on a cork pad. A Acuson Sequoia C256 echocardiography system (Acuson, Mountain View Calif.) was used with a 15.8 MHz probe. Endsystolic (ESD) and enddiastolic (EDD) diameter were measured for the calculation of fractional shortening in a cross section of the heart according to the following formula: (FS) as FS=(EDD−ESD)/EDD.

Bioluminescence imaging (BLI) was performed as follows: rats were anesthetized with 2.5% isoflurane using an XGI-8 anaesthetic chamber designed for use for the IVIS imaging system (Xenogen Corporation, Alameda Calif.), as per the instructions of the manufacturer, and 150 mg/kg luciferin was injected intraperitoneally. After 10 minutes for the luciferin to disperse, images were obtained with an IVIS cooled CCD camera detection apparatus. A grayscale photograph is first obtained with an external light source, and then the source is turned off to obtain the bioluminescence signal, which is then superimposed on the photograph. Images were obtained using five minute integration and a pixel binning of 10, and were processed with LIVINGIMAGE software (Xenogen). Imaging procedures using BLI have been extensively described (Contag, P. R. et al. (1998) Nature Medicine 4: 245-247).

Following echocardiography, the hearts were harvested and analyzed for GFP activity and analyzed with antibodies against cardiac/muscle markers (connexin 43 and α-sarcomeric actin). Hearts were excised and fixed in 2% paraformaldehyde in PBS for 2 hours and cryoprotected in 30% sucrose overnight at 4° C. Tissue was embedded in OCT medium and sectioned at 6 μm on a cryostat. Serial sections were stained with hematoxylin and eosin, Masson's trichrome, or used for immunohistochemistry. Immunostaining was performed as herein described. Briefly, sections were blocked and incubated with primary antibody for 30 minutes to 8 hours at ambient temperature. Primary antibodies against cardiac, human nuclear proteins, and GFP proteins were used. These included rabbit anti-connexin-43, mouse monoclonal anti-α-sarcomeric actin, (Sigma, St. Louis Mo.), mouse anti-human nuclear antigen (Chemicon, Temecula Calif.), goat anti-GFP antibody (Rockland, Gilbertsville Pa.), and rabbit anti-GFP Alexa-488 conjugated antibody (Molecular Probes, Eugene Oreg.).

After brief washing with PBS, sections were incubated with secondary antibodies for 30 minutes to 2 hour at ambient temperature. Texas red conjugated secondary antibodies were used against the cardiac marker and human nuclear antigen primary antibodies. Goat anti-GFP antibody was recognized by a FITC conjugated secondary antibody. Other sections were stained with rabbit anti-GFP Alexa-488 conjugated antibody or in case of the human nuclear antigen stain, no antibodies were used to enhance the GFP signal. Infarcted animals with and without hESC were used as negative controls for immunohistochemistry. After brief washing with PBS, sections were mounted with Slowfade antifade reagent with 4′-6-diamidino-2-phenylindole.HCl (DAPI; Molecular Probes, Eugene Oreg.). Stained tissue was examined with a Leica DMRB fluorescent microscope and a Zeiss LSM 510 two-photon confocal laser scanning microscope. Connexin 43 and α-sarcomeric actin were used to identify differentiation when expressed on donor, GFP-positive cells. Trichrome and H&E stains were used to estimate extent, distribution, structure and kinetics of ensuing scar after the infarction and injection of cells, the mode of their organization and cellular type. H&E was helpful for evaluating cellular atypia and nuclear polymorphism, as indicators of tumor formation.

Although in this example the ES cells were not incubated with ascorbic acid (or any other free-radical scavenger or anti-oxidant), such compounds may be optionally added to the culture medium as described in Example IV.

FIG. 1 shows that intramural injection of the bioartificial tissue ES cell-containing matrix results in homogenous support of injured myocardium, with alignment along the collagen fibers. Smooth intermingling of donor (red) and host (blue) collagen structures between the epicardial (arrowheads) and the endocardial (arrows) portion of the recipient heart muscle. The gaps between the collagen fibers are filled with donor cells in serial alignment. An inflammatory response with massive cell infiltration, foreign body reaction, and consequent diminishing of the grafted structure was not observed.

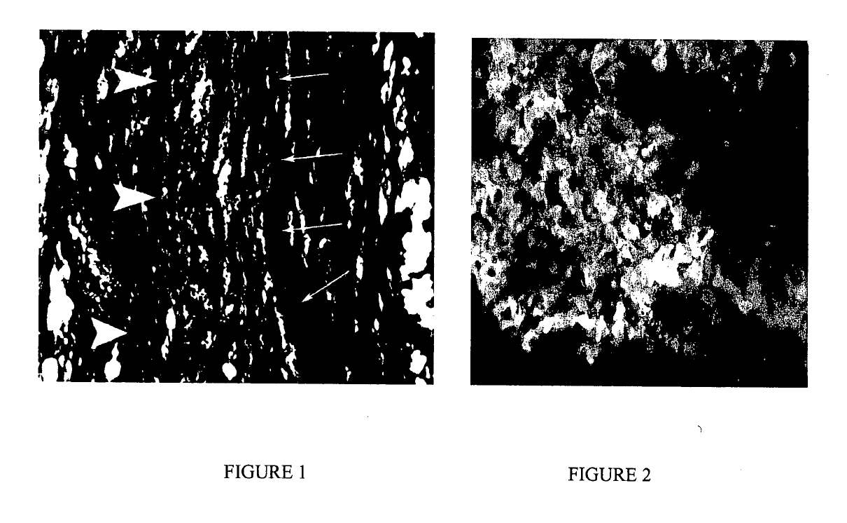

FIG. 2 shows that dense populations of GFP-positive donor cells formed robust grafts within injured myocardium as they co-localized with the blue-staining DAPI nuclear signal and with expression of connexin 43 (red).

As shown in FIGS. 1 and 2, the graft formed a sustained structure within the injured area and prevented ventricular wall thinning. The inoculated cells remained viable and were shown to express connexin 43 and α-sarcomeric actin.

Fractional shortening (measured as percentage (%) decrease of the ventricular lumen) and regional contractility were better in animals which received bioartificial tissue grafts compared to the controls (infarcted, matrix-only, and ES cells-only: Group I (transplanted healthy heart): 17.0±3.5%, Group II (infarcted control, no ES cells, no matrix): 6.6±2.1%, Group III (matrix alone): 10.3±2.2%, Group IV (ES cells plus matrix): 14.5±2.5%, Group V (ES cells alone, no matrix): 7.8±1.8%).

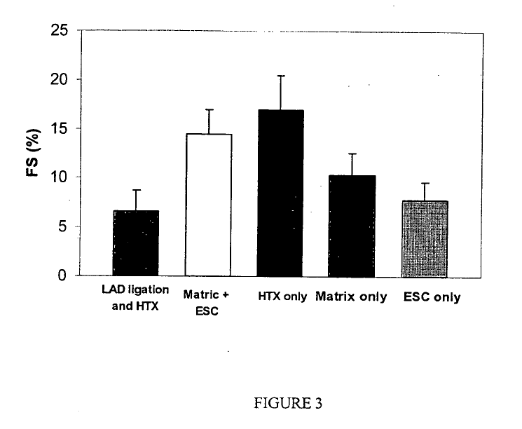

The data are presented in graphical form on FIG. 3. The legends correspond to the above Groups as follows: LAD ligation and HTX (Group II); Matrix and ESC (Group IV); HTX only (Group I); Matrix only (Group III); and ESC only (Group V).

From analysis of all the data it was concluded that the liquid bioartificial tissue containing ES cells constituted a powerful new approach to restore injured heart muscle without distorting its geometry and structure.

### Example II

### Preparation and Introduction of ES-Derived Cardiomyocytes into a Large Animal Model of Myocardial Infarction

The ES cells are prepared as described in Example I. A suitable large animal that is routinely used to study clinical procedures used to alleviate myocardial infarction is obtained. An example of such an animal is the pig.

A pig, with a mass of between 30 and 35 kg, is anaesthetized, paralyzed, and prepared for the procedure. The LAD is ligated to create an intramural left ventricular pouch (infarcted area of the myocardium). The ES cells (between 2×10⁶ and 10⁹ cells) are suspended in between 0.125 ml and 1.0 ml MATRIGEL and injected in the resulting infarcted area within the pouch. Five recipient groups are studied: transplanted healthy hearts (Group I), infarcted control animals (Group II), matrix recipients alone (Group III), the study group which receives matrix plus cells (Group IV), and a group which receives ES cells alone (Group V). Two weeks later, the animal is subjected to echocardiography to visualize the extent of colonization by the cardiomyocytes. Following echocardiography, the heart is harvested and analyzed for GFP or other bioluminescence activity and analyzed with antibodies against cardiac/muscle markers (connexin 43 and α-sarcomeric actin) on the same sections using confocal microscopy and 3-dimensional reconstruction as described in Example I.

### Example III

### Preparation and Introduction of ES-Derived Cardiomyocytes into a Human Subject with Myocardial Infarction

A human subject presents with myocardial infarction. The area of injured myocardium is identified using an endoscope device. ES cells are prepared as described in Example I. The ES cells (between 2×10⁶ and 10⁹ cells) are suspended in between 0.125 ml and 1.0 ml MATRIGEL and injected in the injured area of the myocardium. The liquid bioartificial tissue is let to become solid and the human subject is observed for heart function, including echocardiography, blood pressure, and other measurements of heart parameters.

### Example IV

### Use of Adjuvant Ascorbic Acid in Transplantable Cell Cultures

Human ES (hES) cell lines, H1 and H7 (Mummery et al., (2002) supra; Hescheler, B. K. et al. (1997) Cardiovasc. Res. 36:149-162) were initially maintained on feeders in ES medium containing 80% knockout Dulbecco's modified Eagle's medium (KO-DMEM) (Invitrogen, Carlsbad Calif.), 1 mM L-glutamine, 0.1 mM β-mercaptoethanol, 1% nonessential amino acids stock (Invitrogen), 20% Serum Replacement (Invitrogen, Carlsbad Calif.) and 4 ng/ml hbFGF (Invitrogen). The cells were later maintained using feeder-free conditions, herein described. Briefly, feeder-free cultures were passaged by incubation in 200 units/ml collagenase IV for 5-10 minutes at 37° C., dissociated and then seeded onto MATRIGEL-coated plates and maintained in conditioned medium (CM) prepared from primary mouse embryonic fibroblast cultures.

Confluent H7 hES cell cultures were incubated with 0.5 mM EDTA in phosphate-buffered saline (PBS) at 37° C. for 8 min, dissociated, resuspended in CM and replated onto MATRIGEL-coated plates at ˜1×10⁶ cells/cm². The cells were exposed to the supernatant containing lentivirus 24 h after plating. DEAE-dextran at a final concentration of 10 μg/ml and hbFGF at 4 ng/ml were added to the viral supernatant immediately before the transduction. The medium was replaced with CM after 12-18 hr incubation. The CM was supplemented with 100 μg/ml G418 five days after transduction. The cells were split with collagenase IV when confluent, seeded onto MATRIGEL-coated plates and maintained in CM supplemented with G418.

Cardiac differentiation of hES cells was induced through embryonic body (EB) formation in the differentiation medium containing 80% knockout Dulbecco's modified Eagle's medium (KO-DMEM) (Invitrogen), 1 mM L-glutamine, 0.1 mM beta-mercaptoethanol, 1% nonessential amino acids stock (Invitrogen), 20% FBS (Hyclone) as described (Xu et al. (2002) Circ. Res. 91: 501-508). At differentiation day 18-21, differentiated cultures containing beating cardiomyocytes were washed with PBS and incubated with 0.56 units/ml Blendzyme IV in PBS (Roche, Indianapolis Ind.) at 37° C. for 30 min. The cells were then dissociated, resuspended in differentiation medium and loaded onto discontinuous PERCOLL gradients for isolation of cardiomyocytes (Xu et al., (2002) supra). Using a bottom layer of 58.5% PERCOLL and a top layer of 40.5% PERCOLL, most of the cardiomyocytes migrated to the bottom layer of PERCOLL. Cells form this layer were then harvested, washed once with the differentiation medium and once with the differentiation medium without serum, and resuspended in the differentiation medium without serum for transplant studies.

The ascorbic acid treatment was as follows: 24 hrs prior to harvest, the medium of the differentiated cultures was changed from DMEM/20% FBS to DMEM/20% FBS+1 mM ascorbic acid.

Mice were pre-anesthetized in an Isoflurane inhalation chamber and received an intraperitoneal injection of ketamine (25 mg/kg). The animals were then intubated and ventilated for the entire length of the procedure. The surgical approach involved a left lateral thoracotomy, pericardectomy and identification of the Left Anterior Descending Artery (LAD) for ligation.

Once ligation with an 8.0 Ethilon stitch (Ethicon, Johnson & Johnson, Sommerville N.J.) was performed on the proximal 2 mm portion of the LAD, a pale area demarcated on the surface of the left ventricle. Placement of the ligature in the basic third of the LAD resulted in significant left ventricular ischemia which was soon (within minutes) irreversible and encompassed the middle and apical portion of the ventricle. This area constitutes the target for the cells. Using a 28 G needle, 250,000 hES cells resuspended in 25 μl of medium were injected into the demarcated area which consecutively became yellowish, a reliable sign that cells have been administered intramyocardially and did not accidentaly enter the left ventricular cavity. Immediately thereafter, a chest tube (16G Angiocath, Beckton Dickinson, Franklin Lakes N.J.) was inserted and the chest closed in layers. Ventilation was maintained until sufficient spontaneous breathing occurred and extubation followed. The mice were left to recover in a temperature controlled chamber, until they resumed full alertness and motility. Individual animals were identified by ear tagging. Animals were sacrificed at various time points after cell transfer and echocardiography in deep anesthesia.

Cells formed conglomerates within the injured myocardium following transplantation, therefore it was not possible to count single cells.

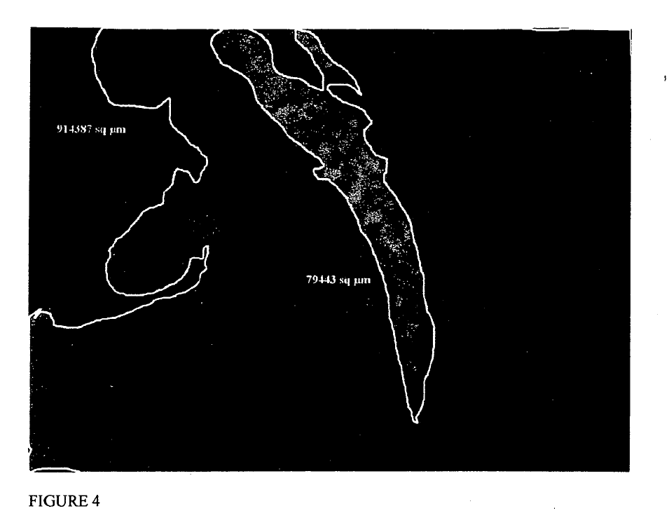

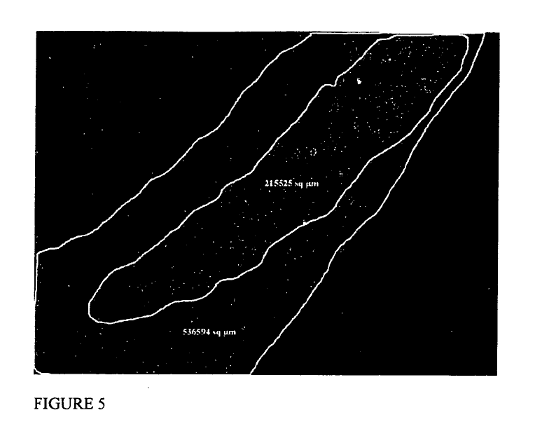

As shown in FIG. 4, six hours after transplantation, in the animals that had been transplanted with untreated cells, only tiny islets of cells were visible, comprising approximately ten cells each. The area of the tissue populated by any of the transplanted cells was measured in the image as 79,443 μm² (delimited by the yellow line) compared with a total area of damaged tissue of 914,387 μm² (delimited by the white line). This resulted in a graft/infarct ratio (R) of 0.09:1.0.

As shown in FIG. 5, 24 hours following transplantation, in the animals that had been transplanted with ascorbic acid-treated cells, huge conglomerates were visible that restored and populated a large proportion of the damaged tissue. The area of the tissue populated by any of the transplanted cells was measured in the image as 215,525 μm² (delimited by the yellow line) compared with a total area of damaged tissue of 536,594 μm² (delimited by the white line). This resulted in a graft/infarct ratio (R) of 0.4:1.0, a 4.6-fold increase compared with the ratio from the above experiment using untreated cells (FIG. 4; R=0.09:1.0).

Similar results were obtained when the transplanted tissues were examined at subsequent time points: 7 days, 14 days, and 28 days following transplantation. The effects on colonization and viability of cells that were pretreated with ascorbic acid was sustained.

### Example V

### Transplantation of Murine ES Cells into Mouse Heart with Myocardial Injury

Plasmid vector pEF-1 a-EGFP (Green Fluorescent Protein), which contains EGFP gene under the control of human EF1 a promoter and a neomycin resistance cassette, was constructed as follows. The promoter region of plasmid vector pEGFP-N3 (Clontech, Palo Alto, Calif.) was removed by removing the Ase I-Nhe I DNA fragment and joining of blunt-ended termini. Human EF1a promoter from pEF-BOS site of the plasmid. ES cells from mouse cell line D3 were transfected with pEF-1 a-EGFP and one clone, brightly expressing EGFP, was chosen and used for the experiments. The clone was adapted to feeder-free conditions and mixed with liquid, growth factor-reduced MATRIGEL (BD Bioscience, Bedmord Mass.). MATRIGEL has the physical property of consolidating to a gel-solid consistency at 37° C. in a few hours. The mixture was injected intramyocardially into the area of acute ischemia, following ligation of the LAD.

Animal Groups: group I (n=5): BALB/c mice received 50 μl of the mixture into the area of myocardial injury. Group II (n=5) underwent LAD ligation only (n=5). In Group III, MATRIGEL alone was injected into the area of myocardial injury (n=5). In Group IV, only ESC were injected (n=5).

Animal anaesthesia: Mice were pre-anesthetized with isoflurane and received an intraperitoneal injection of Ketanest/Xylazine (50 mg/kg body weight). The animals were then intubated and ventilated for the entire length if the procedure. The surgical approach involved a left lateral thoracotomy, pericardectomy (mouse pericardium is a transparent membrane and removing it or incising it is inevitable if a cardiac procedure is intended), and identification of the left anterior descending artery (LAD) for ligation. Once the pericardium was opened in a mouse, the left atrium could be seen contracting vigorously.

Surgical methodology: From a lateral approach, one looks for the middle of the free margin of the left atrium. This is the point the surgeon usually identifies the LAD, and moves distally to the transition from the 1^{st} to the 2^{nd} third of the vessels course on the surface of the LAD. This can be viewed as the optimal spot for the LAD ligation, in order to obtain a significant infarction of the mentioned magnitude. Ligation in the immediate proximity of the left atrial margin (too proximal) usually causes death of the animal. A ligation further distally will cause a much to small infarction that will not impact left ventricular function sufficiently.

Following ligation of the LAD, 10⁶ donor ES cells in 25 μl medium were mixed with 25 μl into liquid MATRIGEL.

Injections: The resulting total compound volume was 50 μl. The injection was targeted into the area of injury that bleaches out immediately following ligation of the LAD in the mouse. Our experience has shown that the mouse left ventricle suffers an infarction of the magnitude of 40-50% of the left ventricular wall by this approach. The transplantation of the liquid bioartificial tissue was then performed on the beating heart of the mouse (during inhalational anesthesia, the heart rate of a BALB/c mouse range between 300-400/minutes). During targeted injection, the affected area swells slightly, an indication that the compound remains intramurally and does not escape into the left ventricular cavity.

Echocardiography: Mice were transferred in a portable anaesthesia chamber and kept under inhalational isoflurane anesthesia for the duration of the echocardiogram performed, which took place immediately before sacrifice. The Acuson Sequoia C256 echocardiography system (Acuson, Mountain View Calif.) with a 15.8 MHz probe was used. The following measurements were obtained: End-systolic (ESD) and end-diastolic (EDD) diameter in a cross section, ESD and EDD at two different sites of a longitudinal section of the heart (basal at the submitral level, and apical), posterior and septal wall thickness (PWT and SWT, respectively), and calculated fraction shortening (FS) as FS=(EDD−ESD)/EDD.

Histology and Immunohistochemistry: The myocardium was sectioned at 5 different transversal levels at the site of tissue necrosis, encompassing the entire lesion. On every single section the infracted area, which can be distinguished easily, both with trichrome stain as well as in the immunofluorescence stains (dead myocardium appears dark, without nuclei (colocalization with DAPI), as opposed to intact myocardium which is rich in nuclei and cell shapes are clearly visible in the green filter). Using the aforementioned area analysis program the entire area of infarction in μm² was measured morphometrically. In mice that received cells, we measured the green fluorescent area, which corresponded to the dense cellular colonies seen. The ratio between the green (graft) area and the infarct area was calculated and which was termed the “graft/infarct area ratio” and expressed this in terms of a percentage (%). These measurements were performed five times for every single animal and the mean was calculated.

Staining and Cytology: 5 μm cryosections were stained with hematoxylin and eosin, Masson's trichrome, or were used for immunohistochemistry. Immunostaining was performed as described by Balsam et al. (Balsam et al., (2004) Nature 428: 668-673). The antibody used was rabbit anti-connexin-43 (Sigma, St. Louis Mo.), goat anti-GFP antibody (Rockland, Gilbertsville Pa.), and rabbit anti-GFP Alexa-488 conjugated antibody (Molecular Probes, Eugene Oreg.). Stained tissue was examined with a Leica DMRB fluorescent microscope and a Zeiss LSM 510 two-photon confocal laser scanning microscope.

Morphometry: For all morphometric evaluations, the focused microscopic field was photographed by an adapted camera (Diagnostic Instruments Inc, Sterling Heights Mich.). The total GFP positive area was measured and related to the infarction area at low magnification (ratio in %). To quantify the degree of expression of specific markers, five random sections of the GFP positive graft were photographed and evaluated using the Spot advanced software, version 3.4.2 (Diagnostic Instruments Inc, Sterling Heights Mich.).

Statistics: All results were expressed as mean±SD. Data were compared, and between-group differences were analyzed by one-way ANOVA with post hoc Bonferroni test. Statistical analyses were performed with STATVIEW 5.0 (SAS Institute, Cary N.C.), and significance was accepted at p<0.05.

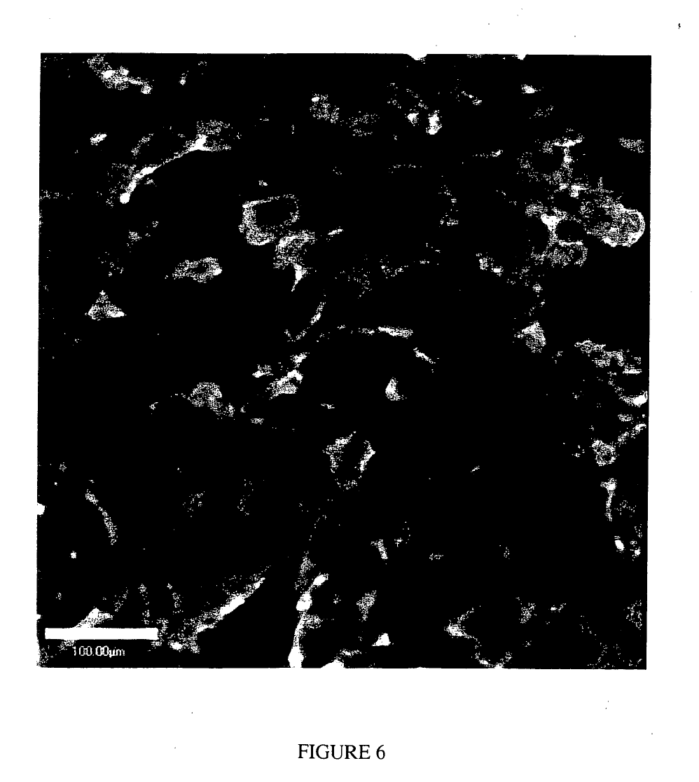

Cells embedded in liquid MATRIGEL formed GFP-positive colonies within the infarcted area and connexin 43 expression (a marker of cardiac differentiation; gap junction formation) was detected (small red spots) at various intercellular contact sites to neighboring cells of both donor and host (see FIG. 6). Signs of cellular atypia, nuclear polymorphism, or teratoma formation were not observed in this group.

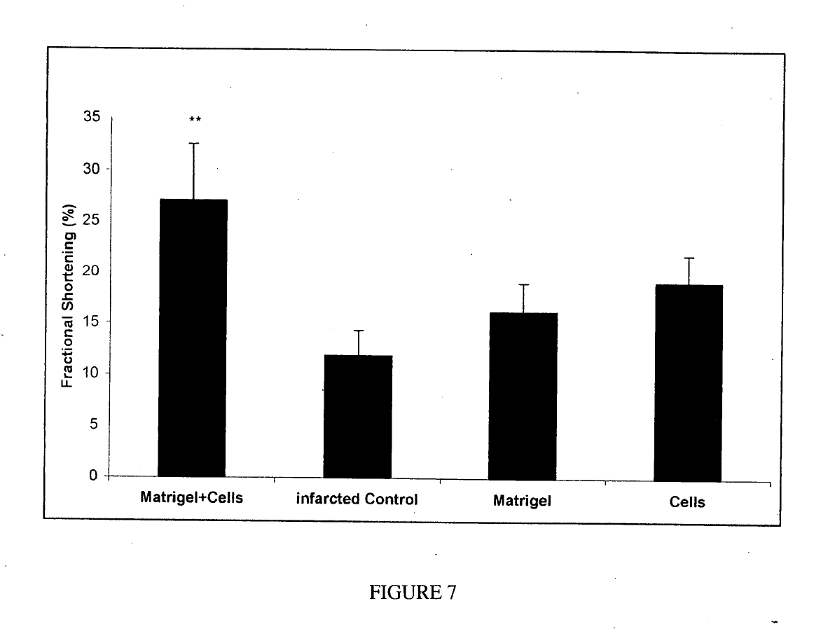

Cardiac Function: Echocardiography revealed superior heart function in the mice that were treated with the liquid compound compared to the controls (F: 16.40, p<0.0001, one-way ANOVA). The hearts' fractional shortening (FS) in the treated and control groups were as follows: group I, 27.1±5.4; group II, 11.9±2.4; group III, 16.2±2.8; group IV, 19.1±2.7 (see FIG. 7). The group treated with cells only (group IV) also showed a significantly higher FS compared with the control group with LAD-ligation and the control group that received only MATRIGEL (p<0.05, Bonferroni post hoc test).

A shown in FIG. 7, the superior fractional shortening results from mice in Group I indicated a superior efficacy of the restorative injection when matrix and cells are injected simultaneously.

Lateral wall (the site of injection) thickness was 0.8±0.05 mm in Group I, 0.5±0.06 mm in Group II, 0.7±0.1 mm in Group III, and 0.7±0.06 mm in Group IV. The lateral wall was the thinnest in the infracted group of mice that did not receive any further treatment (p<0.05, F: 22.49). Similarly, Septal Wall Thickness was 0.7±0.05 mm in Group I, 0.4±0.03 in Group II, 0.65±0.05 in Group III, and 0.67±0.06 in Group IV. Again, septal wall was thinnest in the infarcted group of animals, which did not receive any further treatment (p<0.5, F: 19.56).

The above is just one example of the use of ascorbic acid to enhance viability of transplanted ES cells, and this method can be modified and used with other cell types, as well as with the other methods described by any examples described in this disclosure. Although this example employs ascorbic acid, various other substances or combinations of substances may be used instead of or in addition to ascorbic acid, such as vitamin E, any anti-oxidants, any free-radical scavenging species, or α-lipoic acid.

### Example VI

### Use of Other Adjuvants Compared with Ascorbic Acid in Transplantable Cell Cultures

In separate experiments, treatment with other adjuvants or treatments were compared with treatment with ascorbic acid. The data are disclosed below.

$$\begin{matrix}No treatment \text{:} & R = < 1 \% of infarcted region . \\ Erythropoetin & R = 5.4 + \text{/} - 3.2 \% of infarcted region \\ Heat \text{-} shock of hES cells & R = 7.2 + \text{/} - 2.6 \% of infarcted region \\ Ascorbic acid & R = 38.5 + \text{/} - 8.1 \% of infarcted region\end{matrix}$$

The data show that treatment of cells with ascorbic acid prior to transplantation into a target organ resulted in up to at least 47-fold increase in transplant cell viability and colonization. This compares with a decrease of viable cells following transplantation as is well known to those of skill in the art. The above data are examples of unexpectedly superior results using ascorbic acid as adjuvant.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. A bioartificial tissue comprising treated stem cells in a liquid matrix, wherein the liquid bioartificial tissue can be introduced into an injured target organ to provide a graft and wherein the stem cells have increased viability compared with untreated stem cells.

2. The bioartificial tissue of claim 1 wherein the stem cells differentiate into cells appropriate for the target organ.

3. The bioartificial tissue of claim 1 wherein the liquid matrix further comprises a protein selected from the group consisting of collagen, fibronectin, actin, vitronectin, members of the laminin, tenascin, and thrombospondin families, and proteoglycans.

4. The bioartificial tissue of claim 3 wherein the protein is selected from the group comprising collagen and fibronectin.

5. The bioartificial tissue of claim 1 wherein the stem cells are selected from the group consisting of embryonic stem cells, neonatal stem cell, adult stem cells, and cardiomyoblasts.

6. The bioartificial tissue of claim 5 wherein the stem cells are selected from the group consisting of embryonic stem cells and cardiomyoblasts.

7. The bioartificial tissue of claim 6 wherein the stem cells are embryonic stem cells.

8. The bioartificial tissue of claim 6 wherein the stem cells are cardiomyoblasts.

9. The bioartificial tissue of claim 7 wherein the embryonic stem cells are selected from the group consisting of human embryonic stem cells, non-human primate embryonic stem cells, porcine embryonic stem cells, caprine embryonic stem cells, ovine embryonic stem cells, rodent embryonic stem cells, and mouse embryonic stem cells.

10. The bioartificial tissue of claim 9 wherein the stem cells are human embryonic stem cells.

11. The bioartificial tissue of claim 1 wherein the liquid bioartificial tissue is introduced into the injured target organ by injecting the liquid bioartificial tissue into the organ.

12. The bioartificial tissue of claim 1 wherein the injured target organ is selected from the group consisting of the heart, liver, kidney, brain, bone, reproductive organs, abdominal tissue, and vascular tissue.

13. The bioartificial tissue of claim 12 wherein the injured organ is the heart.

14. The bioartificial tissue of claim 1 wherein the injured target organ is a heart having a myocardial infarction.

15. The bioartificial tissue of claim 1 wherein the bioartificial tissue is liquid.

16. The bioartificial tissue of claim 1 wherein the liquid matrix further comprises a composition selected from the group consisting of growth factors, angiogenic factors, antioxidant, and nutrients.

17. The bioartificial tissue of claim 1 comprising stems cells further having been pre-incubated for a period of between about 4 and 24 hours with a defined cell culture medium formulation comprising a compound having biological activity that increases the viability of the stem cells in the target organ.

18. The bioartificial tissue of claim 17 wherein the compound in the defined cell culture medium is an anti-oxidant.

19. The bioartificial tissue of claim 18 wherein the antioxidant is ascorbic acid.

20. A method of using the bioartificial tissue of claim 1 to treat ischemic heart damage following myocardial infarction, the method comprising the steps of:
(i) providing the bioartificial tissue of claim 1, and
(ii) injecting the cell culture matrix into the ischemic heart; the method resulting in a treated heart.

21. The method of claim 20 further comprising a step of exposing the stem cells to ascorbic acid prior to step (i).

22. The method of claim 21 wherein the concentration of ascorbic acid is between about 0.01 mM and about 10 mM.

23. The method of claim 22 wherein the concentration of ascorbic acid is between about 0.05 mM and about 5 mM.

24. The method of claim 23 wherein the concentration of ascorbic acid is between about 0.1 mM and about 1 mM.
